(19) United States
(12) Patent Application Publication
(10) Pub. No.: US 2010/0089234 A1
(43) Pub. Date: Apr. 15, 2010

(54) Device and Method for Separating Particulate Material from a Gas Flow

(75) Inventor: Eliane Khoury, Delft (NL)

Correspondence Address: Law Offices of Daniel L. Dawes;Dawes Patent Law Group, 5200 Warner Blvd, Ste. 106, Huntington Beach, CA, 92649, US

(73) Assignee: Virus Free Air B.V., Delft (NL)

(21) Appl. No.: 12/444,844

(22) PCT Filed: Oct. 11, 2007
(86) PCT No.: PCT/NL07/00259

§ 371 Date: Jun. 5, 2009

(30) Foreign Application Priority Data
Oct. 11, 2006 (NL) 1032666

(51) Int. Cl. B03C 3/34; B03C 3/49; B03C 3/15; B03C 3/36; A61L 9/00; A61L 9/20; B03C 3/019

(52) U.S. Cl. 95/81; 95/57; 96/83; 96/60; 96/61; 96/74; 96/55; 96/75; 422/187

(57) ABSTRACT

The present invention relates to a device and method for separating particulate material from a gas flow, comprising a substantially elongate housing provided with a peripheral wall and an inlet opening for admitting the gas flow with particulate material into the housing; a number of electrodes extending in the housing up to a predetermined radial distance from the peripheral wall; and an outlet opening arranged downstream in the housing for discharging the gas flow, wherein the outlet opening extends from the axial centre line of the housing to a point some radial distance from a peripheral wall of the housing, and wherein the particulate material is guided into an intermediate space between the outlet opening and the peripheral wall.

The present invention relates to a device and method for separating particulate material from a gas flow.

With the continuous growth of the world's population, technology and industry, increasingly larger numbers and multiple types of particulate material, also including pathogenic micro-organisms, are generated and released into the air. The number of diseases and the number of outbreaks thereof are increasing as a result. Examples hereof are, among others, the outbreak of the Ebola virus, Foot and Mouth disease and the SARS epidemic. Human contact with and exposure to such pathogenic particulate material in the air is increasing, which results in a further increase in the risk of infection and spread of diseases. The air quality is therefore of particular importance, particularly in environments where the chance of infection is high. An example of such an environment is an operating room, where patients, often with open wounds, are susceptible to aerogenic pathogenic particulate material. The set requirements for the air quality, i.e. the quantity of particulate material present in the air must lie below a determined maximum level, will increase further in the future.

One of the most sensitive market segments for aerogenic pathogenic particulate material and infections resulting therefrom are hospitals, where diseases and bacteria of all types converge and are concentrated at the same location. In order to achieve a high air purity, operating rooms are often connected to an air ventilation system which is equipped with air filters with a high efficiency, such as a ‘High Efficiency Particulate Air (HEPA)’ filter. Even such high-efficiency air filters have a limited efficiency and functional flexibility, and this affects their effectiveness under practical conditions. The efficiency of such HEPA filters thus depends on, among other factors, the flow rate, the pressure, flow speed and the heat or possibly present radiation.

HEPA filters have a high efficiency in respect of filtering a gas flow up to particles with a minimum size of 0.3 micrometre. Many micro-organisms, including viruses, bacteria and protozoa, as well as other particulate material present in the air, are however smaller than 0.3 micrometre. Existing HEPA filters are not therefore suitable for filtering this particulate material smaller than 0.3 micrometre, which has the result that micro-organisms penetrate the existing filters and pose a threat to, among others, patients and doctors.

It also occurs that intensive care facilities, which are sometimes not even provided with high-efficiency filters such as HEPA filters, are connected to the general hospital ventilation system, which also purifies the air of the patients' rooms. In such systems the air can be recirculated through the entire hospital, which results in particulate material being spread through all spaces connected to the ventilation system, possibly even including the intensive care facilities.

Research into the situation in the Netherlands in 2005 by the Nederlands Rijksinstituut voor Volksgezondheid en Milieu (RIVM, National Institute for Public Health and the Environment) has shown that 10% to 30% of patients admitted to a Dutch hospital have hospital-related infections, 30% to 40% of which are caused by aerogenic micro-organisms. Furthermore, statistics show that, compared to other sectors, hospital staff have the highest illness-related absence, measured both in days and in frequency.

In order to reduce the risk of infections, in particular in hospitals, there is a need for a system which is able to separate particulate material from a gas flow. There is a particular need for a system which can remove from the air and, if desired, eliminate particulate material of a biological nature in the form of micro-organisms, which consist mainly of bacteria and viruses. Such micro-organisms are often smaller than other particulate material present in the air, such as dust, and thus form part of the aerogenic particulate material which is very difficult to filter or separate, while forming the greatest health risk to people, animals and plants.

An object of the present invention is to propose a device and method for separating particulate material from a gas flow, wherein the stated problems are obviated and which is particularly suitable for separating from the gas flow smaller particulate material than is possible with existing techniques, thereby further improving the gas quality.

The stated object is achieved with the device for separating particulate material from a gas flow, comprising: a substantially elongate housing provided with a peripheral wall and an inlet opening for admitting the gas flow with particulate material into the housing; a number of electrodes extending in the housing up to a predetermined radial distance from the peripheral wall; and an outlet opening arranged downstream in the housing for discharging the gas flow, wherein the outlet opening extends from the axial centre line of the housing to a point some radial distance from a peripheral wall of the housing, and wherein the particulate material is guided into an intermediate space between the outlet opening and the peripheral wall.

In an embodiment the outlet opening is situated downstream, on the opposite side of the inlet opening.

In this patent the term ‘contaminated gas flow’ is understood to mean the gas flow with particulate material admitted into the housing through the inlet opening of the housing. The term ‘purified gas flow’ refers to the gas flow leaving the outlet opening of the housing. The term ‘particulate material’ is understood in this patent to mean all the particulate material present in a gas flow, including micro-organisms, bacteria and viruses, but for instance also dust particles.

By generating a gas flow through a plurality of generated corona discharges, the gas flow is guided such that it is suitable for separating and/or collecting particulate material present in the gas.

Separate corona discharges ionize the gas molecules and cause a movement of the ionized gas molecules in the direction of the applied electric field. At a sufficiently high potential difference, for instance several kV, a field strength of such a magnitude is generated that ions are formed. When the field strength is sufficiently high, a flow of ions will be realized in the electric field. During the movement of ions in the electric field generated by the field and thermal influences, collisions will occur between the ions and neutrally charged gas molecules, which causes the transfer of electrical charge and momentum between elements. The collisions and charge transfer ensure that a movement of the whole gas flow is brought about in the direction of the electric field. Such a generated gas flow is referred to as “corona wind”, “ionic wind” or “electric wind”, and depends on the applied potential difference and the applied current strength. The field strength is proportionally dependent on the potential difference. The higher the potential difference and/or the current strength, the higher the field strength, and therefore also the flow speed, will be. The generation of a flow by the corona wind makes mechanical pumps or a special design of the housing unnecessary. Due to the absence of moving parts the device moreover requires little maintenance and is furthermore not susceptible to wear. In addition, the absence of a mechanical pump results in a weight reduction.

By arranging a plurality of electrodes a gas flow is generated along the path in which the electrodes are placed. The electrodes are arranged at a mutual distance of for instance 1 mm to 500 mm, whereby the device will comprise many tens of or even hundreds of electrodes. When the electrodes are placed in a curved path, particulate material which is moved by this flow will be exposed at the position of the curves to centrifugal forces which, at a sufficiently high flow speed, ensure that the particulate material is separated in accordance with the separating principle of a cyclone. By placing the electrodes at a suitable mutual distance the corona discharge generated individually between each electrode pair and the corona wind generated thereby are combined with the gas flow generated by an adjacent electrode pair such that a continuous and rapid gas flow is realized. The distance between the electrodes depends on the dimensioning of the overall system and can vary in order of magnitude from several micrometres to several centimetres. By applying a sufficiently high potential difference a flow speed is obtained wherein the particulate material moves in outward radial direction toward the wall as a result of the transverse acceleration. The particulate material which displaces toward the wall of the housing as a result of the transverse forces exerted on the material will be located at a continually increasing radius inside the housing during movement through the housing. At a sufficiently high centrifugal force particulate material will become situated at a continually increasing radius relative to the central axis during movement through the housing. A part of the particulate material which collides with the wall of the housing under the influence of the centrifugal force will adhere to the wall. As a result of the centrifugal force particulate material which does collide with the wall but does not adhere thereto will remain in the vicinity of the wall and be moved along the wall with the flow. In short, a part of the particulate material will adhere to the wall or will be displaced at least wholly or partially in the direction of the wall. The gas flow located in the centre of the housing will comprise the lowest concentration of particulate material and will therefore be the best purified. The outlet opening is therefore preferably placed such that it begins at or close to the centre of the housing and extends to a point some radial distance from the wall of the housing. The closer toward the centre and the greater the distance from the wall, the less particulate material there will be present in the air flow leaving the housing through the outlet opening. In the case of a tubular housing the discharge channel is preferably arranged on the central axis of the housing, and the discharge channel has a radius which is at least smaller than the radius of the housing.

In an preferred embodiment the electrodes comprise sharply pointed shapes of electrically conductive material. Sharply pointed shapes have the property of concentrating a charge. The intensity of the field strength around a surface of an electrically charged conductor increases as the radius of curvature decreases and the surface takes on sharper forms. The positive and/or the negative electrodes therefore have a pointed or sharp form.

In a preferred embodiment the electrodes are needle-like. The needle form comprises at least a tubular part with an outer surface and a pointed outer end.

In yet a further preferred embodiment each needle-like electrode is adapted to receive a corona discharge with an outer surface and to generate a corona discharge to an outer surface of an adjacent electrode using a pointed outer end. The electrodes function on the one hand as discharge electrode and on the other as receiving electrode, or counter-electrode. Because for each electrode the subsequent electrode functions as a counter-electrode, additional earthed surfaces can be dispensed with.

In a further preferred embodiment of the invention the electrodes are arranged in a helical line. It is possible to envisage the electrodes being arranged in a number of circles, although it is recommended to arrange the electrodes in a helical line. A helical line has the additional advantage that both a radial and an axial flow is generated through the housing, whereby no additional gas flow-generating means such as a pump are necessary. A helical line is understood in this patent to mean both an ascending line on a cylindrical plane and an ascending line on a (truncated) conical plane.

Although it is possible to envisage the housing being manufactured from a material with a low electrical conductivity, such as for instance plastic, for instance polyethylene, in a further preferred embodiment the housing is manufactured from non-static material. A suitable non-static material is for instance glass, and more particularly quartz glass. Glass has the further advantage that, due to its transparent properties, it is possible to look into the housing from outside and thus monitor whether the device is operating properly and/or may require maintenance and/or cleaning operations.

In a further preferred embodiment the power supply brings the electrodes to a potential difference in the order of magnitude of 0-50 kV. The magnitude of such a potential difference depends on the application; preferred ranges are 10-40 kV, 20-30 kV and about 20 kV, and the value 2, 3, 4, 5, 6, 7, 8 and 9 between 1-10 kV, subject to the application.

In yet a further preferred embodiment adjacent electrodes have a mutual potential difference. The potential difference is preferably applied in each case between two adjacent electrodes. The direction of the flow generated during the corona discharge depends on the polarity of the generated ions and the potential difference of the field. Owing to a potential difference applied in controlled manner the corona discharge can take place in controlled manner between different pairs of electrodes. A gas flow is generated by causing the corona discharge to take place between a continuously changing pair of electrodes. It is possible to envisage a number of electrodes being earthed.

Because the polarity of the corona can be positive as well as negative, different contaminants present in the gas flow, which themselves include a positive or negative charge, can be selectively repelled or attracted. Bacteria usually have a negative potential.

The electrodes comprise a negative or positive potential, or are earthed, wherein adjacent electrodes form electrode pairs. Different potential differences can be applied between different electrode pairs, thereby creating a potential gradient which makes it possible to generate an extra acceleration of the gas flow.

In a preferred embodiment electrodes with a positive polarity and electrodes with a negative polarity are arranged alternately. In this embodiment an electrode with a positive potential has an electrode with a negative potential on either side. The presence of electrodes with a positive potential has the advantage that contaminants with a negative charge—which also includes many bacteria—are attracted hereby. This in contrast to devices which alternately apply an electrode with a negative potential and an earthed electrode. Because contaminants with a negative charge—which also includes many bacteria—are here repelled, an undesired disruption of the gas flow can occur. Due to this disruption the flow might pass from laminar state to an undesired turbulent state.

In a further preferred embodiment a control unit is further provided for applying a potential difference between electrode pairs in controlled manner such that a corona wind is generated. The separating effect will differ for particulate material of different dimensions and mass: the lighter the particulate material, the higher the flow speed, and therefore the applied potential difference, must be in order to move the particulate material outward under the influence of the centrifugal force. The separation can take place selectively by adapting the applied potential difference to the desired particulate material.

In a further preferred embodiment an accumulating unit is arranged in the intermediate space between the outlet opening and the peripheral wall for the purpose of receiving particulate material. As a result of the centrifugal action particulate material will move in outward direction toward the wall and be guided into the intermediate space between the outlet opening and the peripheral wall. Particulate material which collides with the wall but does not adhere thereto will also be entrained by the gas flow through the housing and guided into this intermediate space. By arranging an accumulating unit in this intermediate space the device for separating particulate material from a gas flow can also be used to collect particulate material, including still living micro-organisms. Such a separating function can serve diverse purposes, such as for instance measuring and determining concentrations of particulate material present in a gas flow for the purpose of research and development, collecting and feeding back valuable particulate material to a process for reuse—optionally after being treated—and separating specific particulate material for the purpose of research and development. The accumulating unit extends some distance from the wall of the housing in inward direction where it is bounded by the outlet opening, which preferably has an upright partition wall extending to some extent into the housing.

In a further preferred embodiment a flow body is arranged on an infeed side of the housing. This flow body ensures that the supplied gas flow with particulate material is guided into the housing at a radius such that the particles are exposed to a sufficiently great centrifugal force. The flow body preferably introduces the gas flow at a radius which is equal to or greater than the radius of the outlet opening. It is noted that a flow body can also be provided on the side where the outlet opening is situated for the purpose of influencing or maintaining the air flow situation (laminar/turbulent).

In a further preferred embodiment a second outlet opening is arranged in the intermediate space between the outlet opening and the peripheral wall of the housing for the purpose of discharging particulate material. Particulate material guided into the intermediate space between the outlet opening and the peripheral wall of the housing is discharged in this embodiment through a second outlet opening. In this way this particulate material can be discharged and subjected to a further treatment. It is also possible to envisage a device provided with an accumulating unit also comprising an outlet opening in a wall of the accumulating unit. In addition to the possibility of discharging particulate material for further purposes, this also has the advantage that the accumulating unit does not become full.

In yet another preferred embodiment a grid is arranged in the intermediate space between the outlet opening and the peripheral wall. This grid can be used as screen. In addition, a desired potential can be applied to the grid or, conversely, it can be earthed, whereby the grid is adapted to neutralize charged particulate material present in the gas flow.

The grid can on the one hand be arranged substantially in transverse direction in the housing, wherein it extends from some distance from the axial centre line of the housing to the peripheral wall or to a point some distance from the peripheral wall. In addition to having a linear form, the grid can here also have a parabolic form.

The grid can on the other hand be arranged in longitudinal direction of the housing at a predetermined radial distance from the peripheral wall. Particulate material which moves outward from the central axis of the housing will move in the direction of the grid, where it is allowed through or held back by the screening action and/or potential of the grid.

According to another further embodiment, the peripheral wall of the housing has a stepped form, wherein a second part of the housing has a greater radius than a first part, and wherein the grid is arranged at substantially the same radial distance from the central axis as the peripheral wall of the first part. The grid is situated here substantially in the line of the peripheral wall of the first part of the housing.

In a further preferred embodiment the wall of the housing is modified for optimal adhesion of particulate material. By for instance making the wall rough or providing it with other properties which enhance the adhesion effect the quantity of separated particulate material can be increased further in a manner which has a minimal effect on the flow situation. A grid-like material as described above can for instance be arranged on the wall for this purpose.

Particulate material which is moved through the electric field by the ionic wind generated by the corona discharges will itself also have acquired an electrical charge here. By arranging on the wall of the housing elements which have an opposite polarity to this particulate material, the particulate material will be attracted by these elements and adhere thereto.

In a further preferred embodiment a UV lamp is arranged in the housing. By exposing micro-organisms to UV radiation and when a sufficient dose of radiation is applied, the DNA/RNA structure of the micro-organisms will be damaged due to damaging of the double carbon bond in the molecules. The micro-organisms are effectively eliminated in this manner. By exposing the flow of particulate material to UV radiation as it passes through the housing, micro-organisms in the particulate material are eliminated in effective manner. For some applications, such as for instance in hospitals, it is not desirable or even not allowed to kill micro-organisms in alternative ways, such as for instance using chemical agents or ozone. UV light is however suitable for most practical applications, whereby together with the separating principle based on the centrifugal action a particularly efficient separation of particulate material from a gas flow is provided, which can also be applied in for instance hospitals.

In a further preferred embodiment the wall of the housing possesses UV light-reflecting properties. The effect of UV radiation on the micro-organisms is improved further by reflection of the UV radiation. It is also possible for a UV reflector to be arranged on the wall of the housing behind a non-static UV light-transmitting layer. In this embodiment the particulate material is received by the layer which transmits UV radiation.

In yet another preferred embodiment a substance which is toxic to determined micro-organisms is arranged on at least a part of the wall of the housing. By providing the wall with a toxic substance which kills, or at least damages, the relevant micro-organisms which come into contact with the toxic wall, the effectiveness of eliminating specific micro-organisms can be further enhanced. The toxin is arranged on the wall of the housing, which has the advantage that the toxin is located inside the device. It is of course also possible to guide the gas flow leaving the outlet opening of the housing through a filter which comprises toxin for killing micro-organisms which might still be present in the gas flow. Tests still to be carried out will indicate which toxic substances will be recommended for which micro-organisms.

In a further preferred embodiment an additional gas flow is supplied to the surface of the electrodes. Purified gas is preferably used for this additional gas flow, obtained for instance by a partial feedback of an already purified gas flow, i.e. a gas flow from which particulate material has already been separated. This gas flow is preferably supplied to the surface of the electrodes in a peripheral direction corresponding to the peripheral direction in which the path of the corona wind moves, such that particulate material is prevented from adhering to the electrodes. This is particularly important when the device is used to collect particulate material in an accumulating unit, wherein it is desired that as much particulate material as possible enters the accumulating unit. A further advantage of the additional gas flow is a reduction of the necessary cleaning and maintenance operations. If it is desired to collect living micro-organisms, measures such as UV light and toxin are not applied, and the formation of the ozone which is harmful to micro-organisms is prevented by applying only positive corona discharges.

Compared to known devices for collecting particles, such as an inertial impactor or cyclone, the device according to the invention has the advantage that the device can be adjusted in flexible manner for different situations by varying the applied potential difference and the applied current strength. Conventional devices such as a cyclone are limited particularly by their geometry to a specific field of application, this not being the case with the device according to the invention. The device according to the invention moreover achieves a higher efficiency.

In a further preferred embodiment a further additional gas flow is supplied to the wall of the housing in through-flow direction of the housing. This additional gas flow ensures that no particulate material will adhere to the wall of the housing, which is particularly important when the device is used to collect particulate material in an accumulating unit. Because the additional gas flow reduces accumulation of particulate material on the wall, the necessary cleaning and maintenance operations are also reduced, and the possibility of application in a continuous production and/or detection process is also provided. A purified gas flow is preferably used. A wall with adhesive properties is of course preferably not applied in this embodiment. Supplying an additional gas flow to the wall provides particular advantages in the application of a device where the wall of the housing has UV light-reflecting properties. Since less particulate material will now adhere to the walls, an undesirable reduction in the reflecting surface is prevented.

In a further preferred embodiment a membrane filter is arranged in the gas flow leaving the outlet opening of the housing. Particulate material which might still be present in the gas flow can be filtered by applying a membrane filter with small pores, preferably with nano-porosity. Another suitable filter can of course also be applied instead of a membrane filter.

In a further preferred embodiment the membrane filter arranged in the purified gas flow is electrically charged. By applying a potential to the membrane filter which is opposed to the charge of the particulate material which has acquired a charge while flowing through the housing, this particulate material will be attracted by the membrane filter and will adhere thereto. Charging of the membrane filter can for instance take place by arranging metal, electrically charged wires in the membrane filter. If electrodes having a positive and negative polarity are applied in the housing, the membrane filter is preferably earthed. Particulate material with both positive and negative polarity will in this case have a potential difference relative to the membrane filter, whereby it will adhere thereto.

In a further preferred embodiment an active carbon filter is arranged in the gas flow leaving the outlet opening of the housing. An active carbon filter is applied in order to absorb gas constituents and to reduce odour emission. Other suitable media for absorbing gases, liquids and odours can of course also be applied in addition to active carbon.

In another further preferred embodiment a conversion unit for converting ozone to oxygen is arranged in the gas flow leaving the outlet opening of the housing. A by-product which is formed in a corona discharge is ozone. Ozone can kill micro-organisms and bacteria through a chemical reaction in which the ozone (O3) is converted into oxygen (O2). By forming a sufficiently high concentration of ozone in the housing by means of corona discharges, the micro-organisms and bacteria present in the housing are effectively eliminated. A further advantage is that the formation of ozone can take place using the electrodes already present, and that the ozone is moreover formed directly between the gas flow with micro-organisms, this enhancing the reaction between ozone molecules and micro-organisms.

In order to prevent the ozone which has not reacted with micro-organisms entering the purified gas flow, the gas flow leaving the outlet opening of the housing is preferably guided through a unit in which the ozone which is still present is converted to oxygen.

It is noted that, in applications in which the formation of ozone is undesirable, positive corona discharges are preferably applied since less ozone is herein formed than in the case of negative corona discharges.

In a further preferred embodiment the device is further provided with a pump, whereby a higher flow rate can be achieved. The pump is preferably arranged on the feed side of the housing, for instance adjacently of the inlet opening of the housing or in a feed channel, this providing the advantage that the gas flow is only purified after the pump. Any particulate material which might be released as a result of the pumping action is therefore also separated from the gas flow.

The present invention also relates to a method for separating particulate material from a gas flow, the method comprising the steps of:
admitting a gas flow with particulate material into a housing through an inlet opening;
applying a potential difference between electrodes arranged in the housing a power supply;
generating a tangential gas flow by means of a plurality of corona discharges generated between the electrodes, wherein at least a fraction of the particulate material moves beyond the radial position of the electrodes; and
separating and/or collecting the fraction of particulate material which has moved beyond the radial position of the electrodes.

Although the device and method according to the present invention for separating particulate material from a gas flow is elucidated on the basis of an application in hospitals, other applications can also be envisaged, such as clinics, homes for the elderly and care homes, public places such as offices, cinemas, hotels, restaurants, schools, universities, sports centres, museums, and means of transport such as bus, train, metro, boat, and particularly aeroplanes. Particularly when passengers remain together in a small space for a long time—as is for instance the case on intercontinental flights—such a system can reduce the spread of diseases. Other possible applications which can be envisaged are to be found in industry, where there is a need for purified air in for instance clean rooms, the food industry, the fine chemical industry and slaughterhouses. It is further also possible to apply the system for private use, for instance in houses, cars and the like.

It is optionally possible to successively place a plurality of devices for separating particulate material from a gas flow, wherein the devices for separating particulate material from a gas flow have different design parameters, such as dimensions and applied potential differences, in order to further treat a gas flow discharged by a first device in a further device which is set to another particulate material. By applying different dimensions and potential differences, the further device for separating particulate material from a gas flow can be adjusted such that specific particulate material is further separated. In addition to the above serial connection, the system can also be connected in parallel.

An exemplary embodiment is further elucidated in the following description with reference to the drawing, in which:

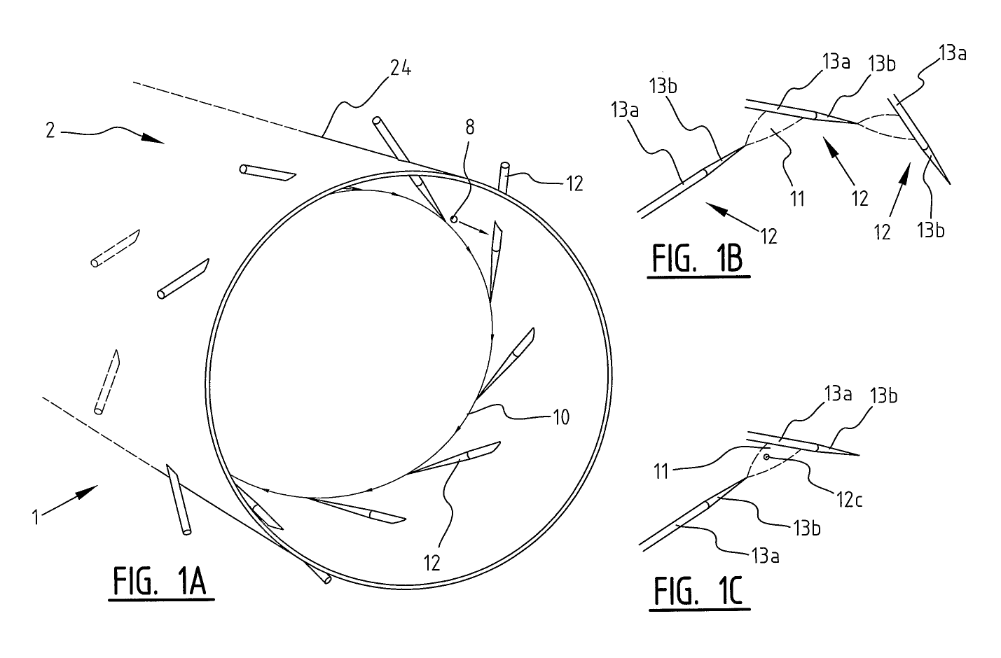

FIG. 1A shows several electrodes arranged in a housing;

FIG. 1B shows a detail view of the electrodes in a first embodiment;

FIG. 1C shows a detail view of the electrodes in a second embodiment;

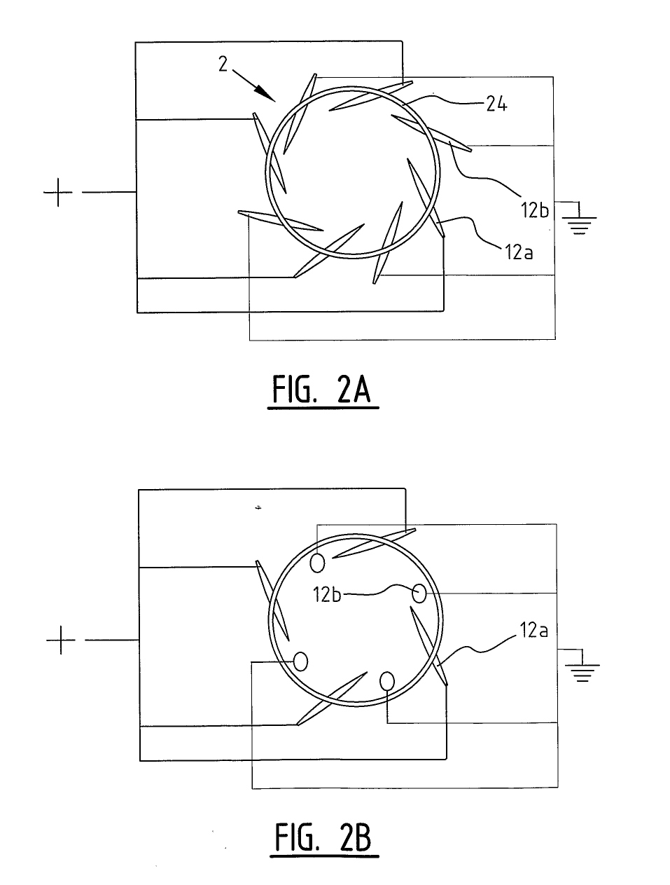

FIGS. 2A and 2B show axial views of the housing with different types of configuration for the arrangement of electrodes;

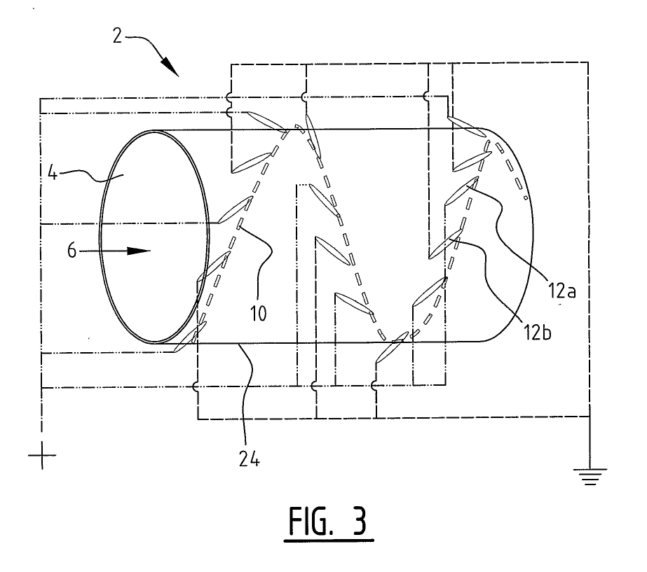

FIG. 3 shows a device for separating particulate material from a gas flow according to a first preferred embodiment of the invention, wherein the electrodes are arranged in a helical line;

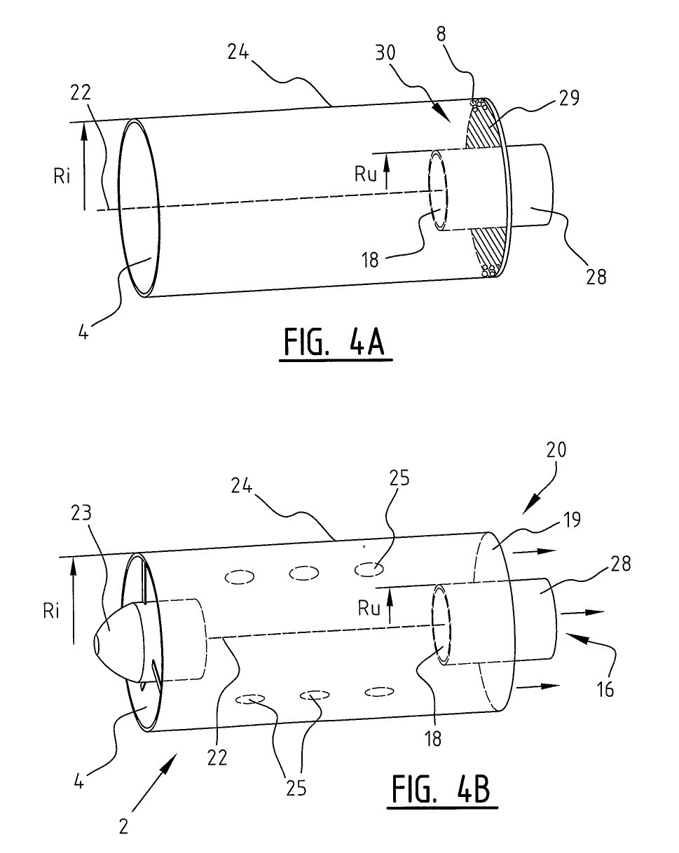

FIG. 4A shows the device of FIG. 3, further provided with an accumulating unit;

FIG. 4B shows the device of FIG. 3, provided with two outlet openings for separate discharge of particulate material and purified gas;

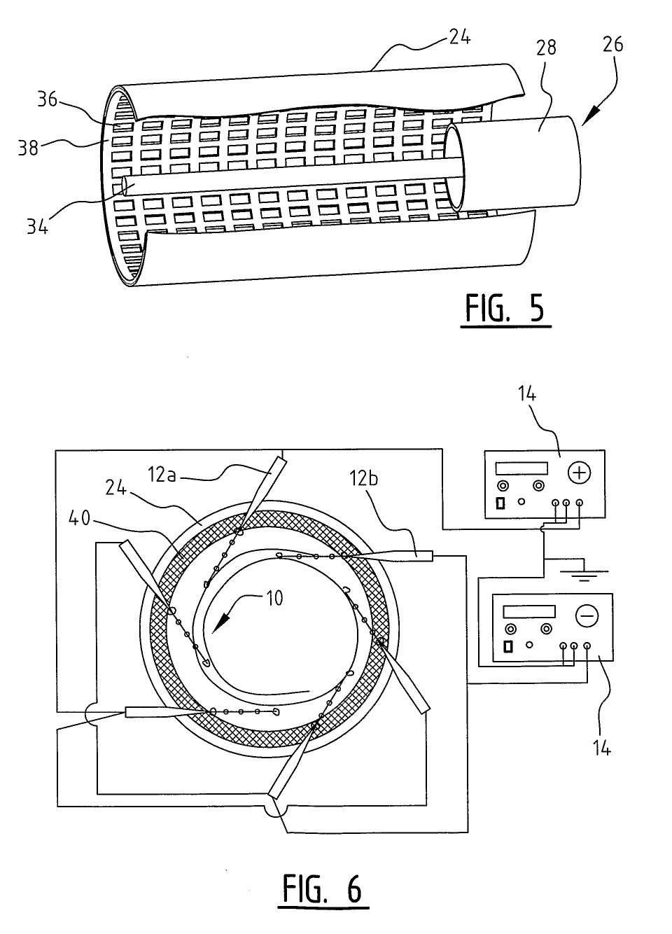

FIG. 5 shows the device of FIG. 4B, further provided with a UV lamp and a UV reflector;

FIG. 6 shows a device for separating particulate material from a gas flow according to a further preferred embodiment, wherein a layer with a toxic substance is arranged on the wall of the housing;

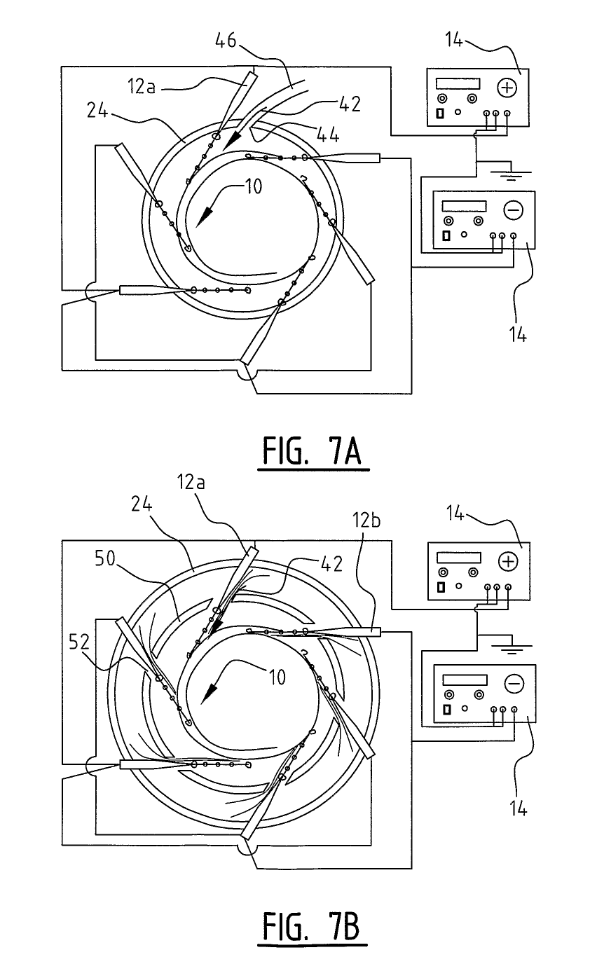

FIG. 7 shows a device for separating particulate material from a gas flow according to a further preferred embodiment, wherein an additional gas flow is supplied to the surface of the electrodes;

FIG. 8 shows a device for separating particulate material from a gas flow according to a further preferred embodiment of the invention, wherein an additional gas flow is supplied to the wall of the housing;

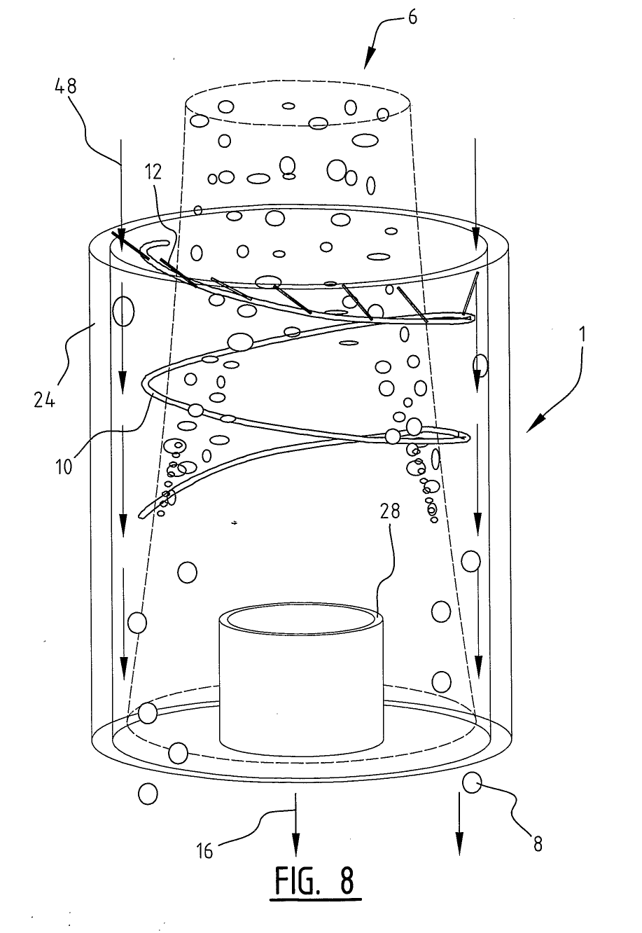

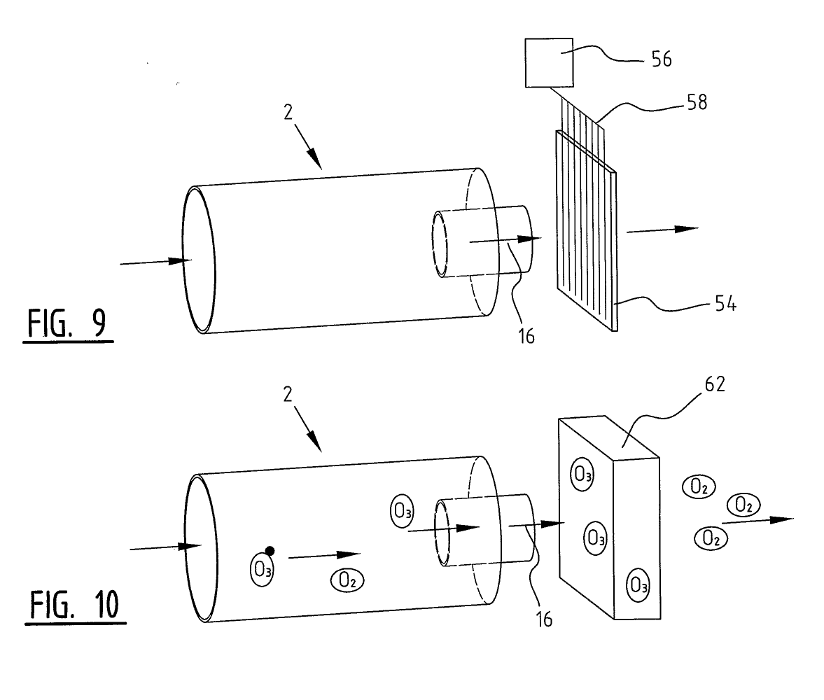

FIG. 9 shows a device for separating particulate material from a gas flow according to a further preferred embodiment of the invention, wherein the gas flow is guided through a membrane filter;

FIG. 10 shows a device for separating particulate material from a gas flow according to a further preferred embodiment of the invention, wherein the gas flow leaving the housing is guided through a unit which converts ozone to oxygen;

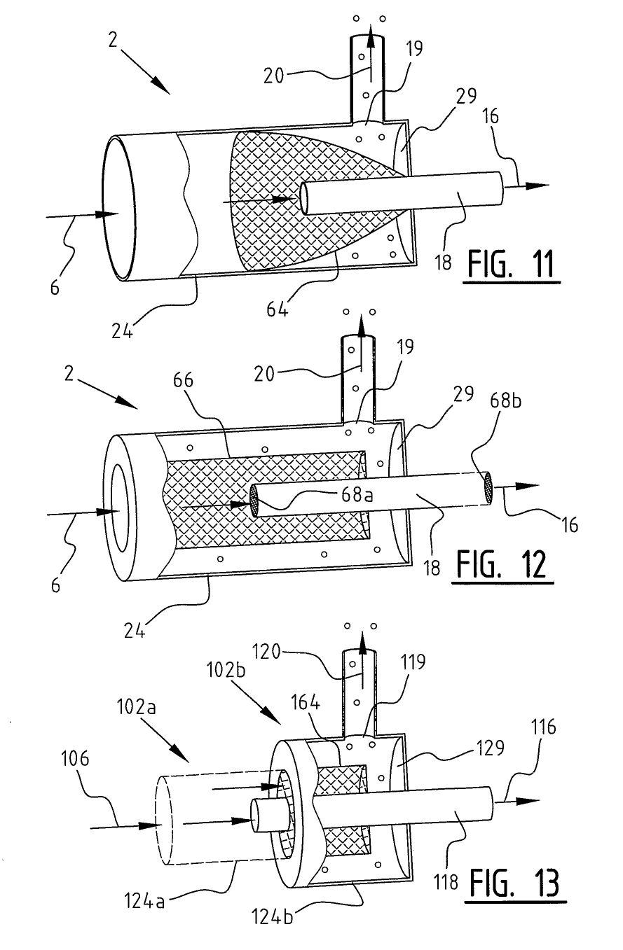

FIG. 11 shows a device for separating particulate material from a gas flow according to a further preferred embodiment, further comprising a grid in a first embodiment;

FIG. 12 shows a device for separating particulate material from a gas flow according to a further preferred embodiment, further comprising a grid in a second embodiment;

FIG. 13 shows a device for separating particulate material from a gas flow according to yet another preferred embodiment;

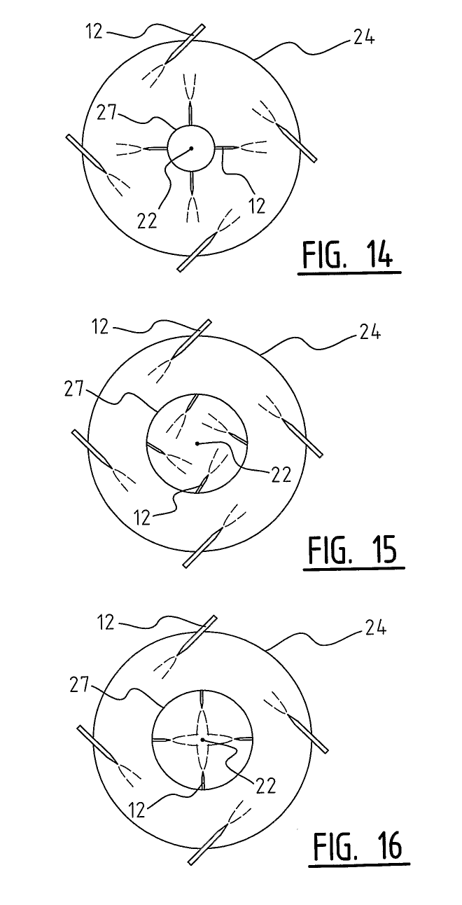

FIG. 14 shows a cross-sectional view of a housing in a further embodiment;

FIG. 15 shows a cross-sectional view of a housing in yet another embodiment; and

FIG. 16 shows a cross-sectional view of a housing in a still further embodiment.

The preferred embodiments as shown in FIGS. 1-16 comprise a device 1 for separating particulate material from a gas flow with a housing 2, provided with an inlet opening 4 and at least one outlet opening 18. A gas flow 6 contaminated with particles 8 is admitted into housing 2 through inlet opening 4, after which the particles 8 are then separated from gas flow 6 in housing 2 so that a purified gas flow 16 results, which leaves housing 2 through outlet opening 18.

According to a first preferred embodiment of the invention, the device for separating particulate material from a gas flow is applied for the purpose of purifying air, for instance in an operating room of a hospital. Such operating rooms are generally connected to an air ventilation system which makes use of the HEPA filters as referred to in the preamble. Device 1 for separating particulate material from a gas flow according to the present invention is optionally placed behind these HEPA filters in the ventilation system. It is also noted that the device for separating particulate material from a gas flow is suitable for providing purified air on its own.

In a preferred embodiment the device 1 for separating particulate material from a gas flow consists of a tubular housing 2 which has on its one side an inlet opening 4 for admitting a gas flow 6 contaminated with particles 8 into housing 2. In housing 2 a number of electrodes 12 are arranged in a helical line 10. A power supply 14 generates a potential difference between electrodes 12. When the potential difference between two adjacent electrodes is high enough, a corona discharge will occur whereby the air molecules located between these electrodes ionize and a so-called corona wind (also referred to as ionic wind or electric wind) is generated. This ionic wind is caused by a movement of the ionized air molecules in the direction of the applied electric field. During the movement of the ions collisions will occur between the ions and neutral gas molecules, which causes the transfer of electrical charges between elements and in this way causes a movement of the whole gas flow in the direction of the electric field along helical line 10. This gas flow depends on the potential difference applied by power supply 14. When the gas flow along helical line 10 has a sufficiently high speed, particles 8 will undergo a centrifugal force in outward radial direction, i.e. in the direction of wall 24 of housing 2. At a determined critical speed the centrifugal force on a particle 8 will be so high that it leaves helical line 10 and moves in the direction of wall 24, and possibly collides with the wall. Housing 2 preferably has a non-static form, for instance by being manufactured from glass.

Electrodes 12 are needle-like and comprise a tubular part 13a with an outer surface and a pointed outer end 13b. Tip 13b of an electrode 12 is directed at an outer surface of a tubular part 13a. The sharp tip 13b has the property of concentrating charge, after which this jumps over via a corona discharge 11 to the outer surface of tubular part 13a at which the tip 13b is directed (FIG. 1B). An additional auxiliary electrode 12c can be arranged between electrodes 12 in order to direct the corona discharges (FIG. 1C).

Once the charge has jumped over to tubular part 13a of the subsequent electrode 12 due to corona discharge 11, the charge will begin to concentrate in tip 13b of this subsequent electrode 12—which electrode 12 can itself also be provided with a potential—and eventually jump over via a corona discharge 11 to the next following electrode 12. Electrodes 12 function on the one hand as discharge electrode and on the other as receiving electrode, or counter-electrode.

The corona discharges taking place between electrodes 12 cause a corona wind which displaces particulate material in the path in which electrodes 12 are placed. As described above, particulate material will be displaced according to a first aspect of the invention by the centrifugal force in the direction of peripheral wall 24 where the material adheres, or is displaced downstream with the flow in the direction of the end of device 1, where the particulate material is separated. According to a second aspect, particulate material will already comprise a determined potential or obtain a determined potential through exposure to the corona wind. According to the second aspect, these particles will adhere to electrodes 12 or peripheral wall 24 with opposite potential. According to this principle, bacteria which by nature are usually negatively charged will for instance adhere to electrodes 12 or a peripheral wall 24 comprising a positive potential.

FIGS. 2A and 2B show different types of configuration for placing of electrodes in a device 1 for separating particulate material from a gas flow. In FIG. 2A the electrodes 12a, 12b have a sharply pointed shape because of the favourable property of sharply pointed shapes for concentrating charge. A potential difference which changes periodically is applied between adjacent electrodes 12a and 12b. FIG. 2B shows an alternative electrode configuration in which a number of electrodes do not have a sharply pointed shape.

FIG. 3 shows a housing 2 which is provided with an inlet opening 4 and in which electrodes 12a, 12b are arranged in a helical line 10. When a contaminated gas flow 6 is set into rotating movement by the corona discharges generated between the electrode pairs placed in helical line 10, the particles 8 located in the contaminated gas flow 6 will move in the direction of wall 24 as a result of the centrifugal force exerted on particles 8. Particles 8 moved in the direction of wall 24 will be situated at a radius greater than the radius of outlet opening 18 when they reach the plane of outlet opening 18, whereby in radial direction these particles 8 are situated between partition wall 28 and peripheral wall 24. These particles 8 will be stopped by rear wall 29 and received in the receptacle 30 formed by peripheral wall 24, rear wall 29 and partition wall 28. Particles 8 will accumulate in this receptacle 30. A discharge conduit (not shown) can optionally be arranged in rear wall 29 of receptacle 30.

In a further preferred embodiment a flow body 23 is preferably arranged on the infeed side of housing 2 in order to ensure that the supplied gas flow 6 with particles 8 is admitted into housing 2 at a radius (preferably equal to or greater than the radius Ru of outlet opening 18) such that the particles are exposed to a sufficiently great centrifugal force, so that an effective separating action is guaranteed. In the unfavourable case that particles 8 are admitted substantially on central axis 22, it is possible to envisage these particles only being set into rotation but, due to the limited radius of the flow field, not moving outward sufficiently in the direction of wall 24 to be removed from the purified gas flow 16 before leaving housing 2. Flow body 23 obviates this problem.

In a further preferred embodiment rear wall 29 is open, thereby creating an outlet opening 19 for discharging a separated fraction 20 of particles 8. By arranging an outlet opening 19 for discharging separated particles 8 no accumulation of particles 8 will take place on the outlet side of housing 2, thereby limiting the required cleaning and maintenance operations.

A number of particles 8 will move so far in radial direction during their passage that these particles 8 come into contact with wall 24 of housing 2. When the collision speed of these particles 8 with wall 24 is sufficiently high, a number of particles will adhere permanently to wall 24. This will particularly be the case with micro-organisms. By modifying wall 24 in a further preferred embodiment such that particles 8 adhere easily to the wall when they collide therewith, the number of separated particles can be increased further. An example of such a modification is to roughen wall surface 24 or to arrange electrically charged elements 25 on the wall surface. By giving these elements 25 a charge which is opposed to the charge of the particulate material in housing 2, this particulate material will adhere to these elements 25 (FIG. 4B).

In a further preferred embodiment of the present invention a UV lamp 34 is arranged in housing 2 (FIG. 5). In the contaminated gas flow 6 a number of the particles 8 will consist of micro-organisms, including viruses, bacteria and protozoa. By exposing these micro-organisms to UV radiation the DNA/RNA structure of the micro-organisms will—with a sufficient dose of radiation—be damaged, whereby these micro-organisms are partially or wholly eliminated. In a preferred embodiment wall 24 is provided with a UV reflector 36, which reflects the UV radiation and thus effectively enhances the effect of the UV light. When it is desired to protect the UV reflector 36 against adhesion of particles 8, UV reflector 36 can be provided with a UV light-transmitting layer 38 on the side of UV lamp 34.

FIG. 6 shows a further preferred embodiment of the present invention, wherein a layer 40 with a toxic substance is arranged on wall 24 of housing 2. When particles 8 move in the direction of wall 24 they will come into contact with toxic layer 40. By applying a substance which is toxic to determined micro-organisms, those micro-organisms sensitive to this toxin can be eliminated.

FIGS. 7A and 7B show preferred embodiments in which an additional gas flow 42 is supplied to the surface of electrodes 12 in order to thus prevent particles 8 adhering to the surface of electrodes 8. FIG. 7A shows a housing 2, the peripheral wall 24 of which is provided with openings 44 connected to a conduit 46. Purified gas, for instance fed back from the purified gas flow 16 which has already passed through device 1 for separating particulate material from a gas flow, is preferably supplied to electrodes 12 through this conduit 46. FIG. 7B shows an alternative configuration in which a preferably purified gas flow 42 is carried in axial direction brought between wall 24 and partition wall 50, and at the position of electrodes 12 flows through openings 52 along the surface of the electrodes so as in this way to prevent adhesion of particles 8 to electrodes 12.

When device 1 for separating particulate material from a gas flow is used for the application of collecting particles 8, for instance for research and development purposes, it is desirable that the number of particles 8 adhering to walls 24, 50 and electrodes 12 is minimal. In a further preferred embodiment of the invention a second additional gas flow 48 is therefore supplied to wall 24 and/or 50 of housing 2, as shown in FIG. 8. Application of this additional gas flow 48 has the further advantage that the adhesion of particles 8 to wall 24 and/or wall 50 is reduced, which further limits the required maintenance and cleaning operations and enables use of a device 1 for separating particulate material from a gas flow according to the present invention in a continuous production process.

The operation of device 1 for separating particulate material from a gas flow according to the present invention can be further improved by further guiding gas flow 16 through a membrane filter 54, wherein this membrane filter 54 is provided in a further preferred embodiment with an electrically conductive wiring 58 which is electrically charged by means of a power supply 56. Particles 8, which have obtained a determined charge as a result of exposure to the corona wind in housing 2 generated by corona discharges, will be attracted to the electrically conductive wiring 58 by applying an opposite charge thereto, and remain in membrane filter 54.

In a further preferred embodiment an active carbon filter 60 is additionally or alternatively applied in order to combat odour emissions.

FIG. 10 shows a device 1 for separating particulate material from a gas flow, wherein an ozone filter 62 is arranged in the purified gas flow 16. A by-product of corona discharges can be the formation of ozone (O3). The ozone formed in housing 2 will react partially with the micro-organisms present in the contaminated gas flow 6 and herein damage or wholly or partially eliminate them. The ozone particle (O3) which reacts with the micro-organism is here converted to oxygen (O2). In order to prevent ozone being present in the gas flow discharged by device 1 for separating particulate material from a gas flow, an ozone filter 62 is placed in the purified gas flow 16 and ensures that ozone particles which have not reacted with micro-organisms are still converted from ozone to harmless oxygen in ozone filter 62.

In a further preferred embodiment a grid 64 is arranged in the intermediate space between outlet opening 18 and peripheral wall 24, which grid functions on the one hand as a screen and can on the other hand, or additionally, be provided with a potential which neutralizes charged particulate material which passes through the grid with the gas flow. In FIG. 11 grid 64 has a parabolic form, although other forms, such as linear, are also possible. In FIG. 11 grid 64 is arranged in housing 2 in transverse direction, wherein it extends in radial direction from outlet opening 18 to peripheral wall 24.

In another embodiment a grid 66 is arranged in longitudinal direction of housing 2 at a predetermined radial distance from peripheral wall 24 (FIG. 12). Particulate material which moves outward from the central axis (not shown) of housing 2 will move in the direction of grid 66, where it is selectively allowed through or stopped by the screening effect and/or potential of grid 66.

Further grids 68a, 68b can be applied per se or in combination in outlet opening 18 for purified gas flow 16. The operation of grids 68a, 68b corresponds with that of grids 64 and 66.

According to an alternative embodiment, peripheral wall 124 of the housing takes a stepped form, wherein a second part 102b of the housing comprises a larger radius than a first part 102a, and wherein grid 164 is arranged at substantially the same radial distance from the central axis (not shown) as peripheral wall 124a of first part 102a. Grid 164 is situated here substantially in the line of peripheral wall 124a of first part 102a of the housing. The separated fraction 120 which has passed through grid 164 leaves the device through outlet opening 119. The operation of the grid corresponds to that of the above described grids 64, 66, 68a, and 68b.

It is noted that in FIGS. 11-13 outlet opening 19, 119 for the separated fraction 20, 120 is not situated in rear wall 29, 129 but in peripheral wall 24, 124b. If desired, a pump can suction the separated fraction 20, 120 to outlet opening 19, 119. In an alternative embodiment outlet openings 19, 119 can of course also be arranged in rear wall 29, 129.

In a further preferred embodiment there is arranged in housing 2 close to central axis 22 a body 27 on which electrodes 12 are provided (FIG. 14). Electrodes 12 on body 27 generate a corona discharge in the direction of electrodes 12 arranged on peripheral wall 24, whereby a centrifugal force directed at peripheral wall 24 is exerted on the particulate material present in the flow. By arranging electrodes 12 at an angle instead of transversely on body 27, the circulation, in addition to an outward directed centrifugal force, is also increased.

In yet another preferred embodiment there is arranged in housing 2 close to central axis 22 a body 27 on which electrodes 12 are provided (FIG. 15). The corona discharge generated by these electrodes increases the circulation of the gas flow in housing 2. Although only one body 27 is arranged in FIG. 15, a plurality of bodies 28 can be provided if desired, wherein the peripheral wall is situated in each case at a different radius relative to central axis 22.

In another further preferred embodiment there is arranged in housing 2 close to central axis 22 a body 27 on which inward directed electrodes 12 are provided (FIG. 16). Using the inward directed electrodes, particulate material present within the volume bounded by body 27 can be provided with an intense charge of a determined polarity. By applying an opposite polarity to peripheral wall 24 the particulate material will then be attracted by peripheral wall 24.

Body 27 may be tubular, but can also comprise a plurality of individual rings placed successively in longitudinal direction of the housing. It is also possible to envisage body 27 comprising an elongate body which is arranged helically in the housing and on which electrodes 12 are arranged. If body 27 is tubular it comprises a porous structure, for instance by embodying body 27 as a grid. It is noted that the body 27 in grid form can also comprise the measures of the above described grids 64, 66, 68a, 68b and 164.

It is also noted that the embodiments shown in FIGS. 14-16 can be combined, wherein the embodiment shown in FIG. 16 is for instance provided with an additional intermediate body as according to FIG. 15.

Device 1 for separating particulate material from a gas flow according to the present invention is able to separate, collect and eliminate particulate material of small dimensions, including micro-organisms such as bacteria and viruses.

While they do show preferred embodiments of the invention, the above described embodiments are intended solely to illustrate the present invention and not in any way to limit the scope of the invention. The scope of the invention is therefore defined solely by the following claims.

1-25. (canceled)

26. Device for separating particulate material from a gas flow, comprising:
a substantially elongate housing provided with a peripheral wall and an inlet opening for admitting the gas flow with particulate material into the housing;
a number of electrodes extending in the housing up to a predetermined radial distance from the peripheral wall; and
an outlet opening arranged downstream in the housing for discharging the gas flow, wherein the outlet opening extends from the axial centre line of the housing to a point some radial distance from a peripheral wall of the housing, and wherein the particulate material is guided into an intermediate space between the outlet opening and the peripheral wall.

27. Device as claimed in claim 26, wherein separate corona discharges ionize gas molecules and cause a movement of ionized gas molecules in the direction of the applied electrical field, wherein collisions between the ions and neutrally charged gas molecules ensure that a movement of the whole gas flow is brought about in the direction of the electric field.

28. Device as claimed in claim 26, wherein the electrodes are placed at a suitable mutual distance, such that the corona discharge generated individually between each electrode pair and the corona wind generated thereby are combined with the gas flow generated by an adjacent electrode pair, such that a continuous gas flow is realized.

29. Device as claimed in claim 26, wherein the electrodes comprise sharply pointed shapes of electrically conductive material, wherein each electrode is adapted to receive a corona discharge with an outer surface and to generate a corona discharge to an outer surface of an adjacent electrode using a pointed outer end.

30. Device as claimed in claim 26, wherein the electrodes are needle-like.

31. Device as claimed in claim 26, wherein the electrodes are arranged in a helical line.

32. Device as claimed in claim 26, wherein the housing is manufactured from non-static material.

33. Device as claimed in claim 26, wherein an accumulating unit and/or a second outlet is arranged in the intermediate space between the outlet opening and the peripheral wall for the purpose of receiving particulate material.

34. Device as claimed in claim 26, wherein a flow body is arranged on the infeed side of the housing.

35. Device as claimed in claim 26, wherein a grid is arranged in the intermediate space between the outlet opening and the peripheral wall.

36. Device as claimed in claim 26, wherein a UV lamp is further arranged in the housing.

37. Device as claimed in claim 36, wherein the wall of the housing comprises UV light-reflecting properties.

38. Device as claimed in claim 26, wherein a substance which is toxic to determined micro-organisms is arranged on at least a part of the wall of the housing.

39. Device as claimed in claim 26, wherein an additional gas flow is supplied to the surface of the electrodes and/or wherein an additional gas flow is supplied in through-flow direction to the wall of the housing.

40. Device as claimed in claim 26, wherein a membrane filter and/or an active carbon filter is arranged in the gas flow leaving the outlet opening of the housing.

41. Device as claimed in claim 40, wherein the membrane filter is electrically charged.

42. Device as claimed in claim 26, wherein a conversion unit for converting ozone to oxygen is arranged in the gas flow leaving the outlet opening of the housing.

43. Method for separating particulate material from a gas flow, comprising the steps of:
admitting a gas flow with particulate material into a housing through an inlet opening;
applying a potential difference between electrodes arranged in the housing a power supply;
generating a tangential gas flow by means of a plurality of corona discharges generated between the electrodes, wherein at least a fraction of the particulate material moves beyond the radial position of the electrodes; and
separating and/or collecting the fraction of particulate material which has moved beyond the radial position of the electrodes.

44. Method as claimed in claim 43, wherein the corona discharges are generated in a controlled sequence.

45. Method as claimed in claim 43, wherein a device for separating particulate material from a gas flow is applied, the device comprising:
a substantially elongate housing provided with a peripheral wall and an inlet opening for admitting the gas flow with particulate material into the housing;
a number of electrodes extending in the housing up to a predetermined radial distance from the peripheral wall; and
an outlet opening arranged downstream in the housing for discharging the gas flow, wherein the outlet opening extends from the axial centre line of the housing to a point some radial distance from a peripheral wall of the housing, and wherein the particulate material is guided into an intermediate space between the outlet opening and the peripheral wall.
